(19) United States
(12) Patent Application Publication
(10) Pub. No.: US 2022/0346370 A1
(43) Pub. Date: Nov. 3, 2022

(54) AN INSULATING MEDICAL DEVICE FOR PROTECTING A GRAFT FOR TRANSPLANT

(71) Applicant: iiSHIELD PTY LTD, Ryde, NSW (AU)

(72) Inventors: Jeremy KWARCINSKI, Ryde, NSW (AU); Henry PLEASS, Hunters Hill, NSW (AU); Tony PANG, Carlingford, NSW (AU); Philip BOUGHTON, Newtown, NSW (AU); Ahmer HAMEED, Eastwood, NSW (AU); Turaab KHAN, Kellyville, NSW (AU)

(73) Assignee: iiSHIELD PTY LTD, Ryde, NSW (AU)

(21) Appl. No.: 17/635,412

(22) PCT Filed: Aug. 14, 2020
(86) PCT No.: PCT/AU2020/050848

§ 371 Date: Feb. 15, 2022

(30) Foreign Application Priority Data
Aug. 15, 2019 (AU) 2019902951

(51) Int. Cl. A01N 1/02; B65D 81/38; B65D 85/00

(52) U.S. Cl. CPC A01N 1/0263; B65D 81/38; B65D 85/54; A61B 2017/00969

(57) ABSTRACT

A medical device for thermally insulating a graft to be transplanted includes a cover body or receptacle. The cover body includes a cavity within which the graft is received, in use. The cover body is made of a biocompatible thermally insulating material. The cover body has a shape similar to that of the graft. In use, the medical device is configured to keep the graft received therein sufficiently cool to substantially prevent warm ischaemic injury to the graft.

### TECHNICAL FIELD

The present invention generally relates to medical devices and in particular to an insulating medical device for protecting a graft for transplant prior to and during the transplant procedure.

### BACKGROUND

Transplantation is the best available treatment for the end-stage kidney failure. Since 2009, the deceased donor rate has increased by over 124%. This has expanded the range of donor kidneys, with approximately 40% donation after death kidneys being transplanted.

When a kidney re-warms during the transplantation procedure before its blood supply has been restored, its subsequent function is impaired (warm ischemic injury), leading to graft (implanted kidney) injury, delayed graft function, prolonged post-operative dialysis and poorer long-term graft outcomes. Yearly dialysis for one patient costs can be in excess of $60,000. The importance of warm ischaemic injury extends beyond its impact on the implanted kidney. The prospect of injury from extended warm ischaemic time often forces the surgeons to perform the procedure quickly. In fact, the greatest cause of graft loss within the first six-months of transplantation is surgical complication, where the blood supply thromboses, and the kidney needs to be removed.

Although currently all kidneys are at risk of this warm ischaemic injury, 50% of circulatory death kidneys experience delayed graft function, with the need for a period of post-operative dialysis. In addition, this injury results in both patient and hospital costs from increased need for dialysis, additional biopsies and testing, longer inpatient stays and poorer overall outcomes.

Kidneys rewarm to over 27° C. during surgical anastomoses. Eurotransplant data shows that for every 10 minutes of anastomosis time, kidney damage occurs, with increased incidence of delayed graft function, biopsy proven fibrosis and poorer 5-year graft survivals. This damage is a direct consequence of kidney re-warming prior to re-institution of the kidney's blood supply in the recipient. In addition, graft thrombosis from technical complications of surgery occurs in between 2-4% in some cases.

Currently in Australia, the US and Europe, there are approximately 45000 kidney transplants a year. Of these, there is an expected failure rate of 10% at 5 years, 20% at 10 years. If we can reduce the failure rate by 5%, we can keep 2000 patients off post-transplant dialysis. With an incremented cost benefit of $40000 per patient, per year.

Furthermore, the capacity to increase the time before warm ischemic injury provides increased capacity for surgical training and a reduced time-pressure on surgeons, providing better outcomes and minimising risk of surgical complication.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

Any one of the terms: “including” or “which includes” or “that includes” as used herein is also an open term that also means including at least the elements/features that follow the term, but not excluding others.

Any discussion of the background art throughout the specification should in no way be considered as an admission that such background art is prior art nor that such background art is widely known or forms part of the common general knowledge in the field in Australia or worldwide.

### SUMMARY

In an aspect, there is provided a medical device for thermally insulating a graft to be transplanted, comprising:
a cover body including an inner surface defining a cavity within which the graft is received, in use, the cover body having an outer edge defining an opening through which the graft is received into the cavity, in use;
wherein the cover body is made of a biocompatible thermally insulating material;
wherein, in use, the medical device is configured to keep the graft received therein sufficiently cool to substantially prevent warm ischaemic injury to the graft.

A shape of the inner surface of the cover body may be similar to a shape of the graft.

The graft may be a transplantable organ selected from the group comprising kidneys, a heart, a liver, lungs, intestine and pancreas.

The cover body may extend over a substantial proportion of an external surface area of the graft, in use.

The cover body may extend over substantially all of an external surface area of the graft, in use.

The cover body may be adapted to closely cover the graft, in use.

The cover body may be adapted to conform to an outer shape of the graft, in use.

The time period may be approximately 60 minutes.

The cover body may be made of a flexible material.

The cover body may be made of silicone.

The cover body may have a substantially uniform thickness.

The thickness of the cover body may be approximately 2 cm.

The thickness of the cover body may be within the range of 1.5 cm to 2.5 cm.

The cover body may comprise an outer surface, the outer surface being textured.

The cover body may comprise an inner surface, the inner surface being textured.

The textured inner surface may be configured to grip an outer surface of the graft, in use, to substantially prevent egress of the graft from the cavity.

The textured surface may comprise a plurality of channels along which cooling fluid can travel to cool an outer surface of the graft, in use.

The textured surface may comprise a pattern including a plurality of interconnected cooling channels.

The cooling fluid may be cold saline.

The medical device may further include a gripping portion extending from the cover body to allow the user to comfortably grip and manipulate the cover body when the graft is located within the cover body, in use.

The medical device may further comprising at least one fastener located on the cover body, adjacent the opening, configured to provide a barrier across the mouth of the cavity to prevent egress of the graft from the cavity, in use.

The at least one fastener may include a first portion attached adjacent a first portion of the edge, and a second portion adjacent an opposing second portion of the edge located across the mouth.

The first portion may comprise a slot and the second portion may comprise a corresponding T-shaped protrusion which releasably engages with the slot in the first portion.

The medical device may be of unitary construction.

The graft may be a transplantable organ.

The graft may be a transplantable organ selected from the group comprising kidneys, a heart, a liver, lungs, intestine and pancreas.

The cover body may further include markings which provide a visual guide to the user, in use.

The medical device of claim 1 may further comprise a cooling pocket for receiving a cooling insert.

The medical device may further comprise a cooling insert wherein the cooling insert comprises saline or where the cooling insert comprise polyurethane.

In another aspect of the present invention, there is provided a medical device for thermally insulating a graft kidney to be transplanted comprising:
a curved cover body having a substantially U-shaped cross-section, the U-shaped cross-section defining a cavity configured to receive and cover the graft kidney, in use;
wherein the curved cover body comprises a biocompatible thermally insulating material and is configured to keep the graft kidney sufficiently cool to substantially prevent warm ischaemic injury to the graft kidney, in use.

The cover body may be curved in an arc about a central axis.

Both arms of the U-shape of the substantially U-shaped cross-section may extend substantially parallel to a plane perpendicular to the central axis.

The cover body may extend over substantially all of an external surface area of the graft kidney, in use.

The cover body may be curved in an arc, thereby defining a recess such that, in use, blood vessels and ureter of the kidney can be positioned within the recess to enable a user to easily access to blood vessels and ureter of the kidney during transplantation.

The cover may include at least one fastener located on the cover body, adjacent the mouth of the cavity, configured to provide a barrier across the mouth of the cavity to prevent egress of the graft from the cavity, in use.

In another aspect of the present invention there is provided, a system for cooling a graft before transplant comprising:
a thermally insulating cover;
   an active cooling apparatus configured to provide cooling to the cover and the graft received therein.

The active cooling apparatus may comprise a thermoelectric cooler.

The active cooling apparatus may comprise an air pump.

The active cooling apparatus may comprise:
cooling pipes located along an inner surface of the cover between the graft;
an inlet for receiving cooling fluid, and
an outlet for removing warm cooling fluid.

The cooling pipes may be in contact with and extend along an outer surface of the cover.

The cooling pipes may be in contact with and extend along an inner surface of the cover.

According to yet another aspect of the present invention, there is provided an insulating medical device for protecting a graft for transplant. The insulating medical device comprises a curved receptacle having an open portion and a closed portion, thereby forming a cavity therebetween. Further, the curved receptacle is adapted to receive a graft in the cavity through the open portion. A shape of the curved receptacle is selected based on a shape of the graft. Additionally, the curved receptacle is made of silicone or another biocompatible insulating material, thereby providing an insulating layer. Moreover, the curved receptacle is configured to keep the graft received therein, within a predetermined temperature range, thereby preventing damage to the graft when the graft is removed from a cold storage for transplantation due to rise in temperature.

The receptacle may be available in a plurality of sizes such as small, medium, large, extra large to suit a variety of different graft sizes. The appropriate size may then be chosen by the surgeon accordingly. The graft may be snug fitting within the receptacle to ensure a good thermal connection to the receptacle. The silicone or other biocompatible insulating material may have elastic properties and may be stretchy to assist snug fitting engagement of the graft within the receptacle.

It is advantageous as the curved receptacle having the silicone insulation helps to maintain the graft at a safe and cool temperature prior to and during transplantation to avoid the warm ischaemic injury occurring during the procedure, prior to revascularization. Potential benefits therefore may include increase short- and long-term function, increase patient quality of life, reduce the pressure for rapid surgery (opening the door to robotic transplantation surgery and improved surgical training) and reduce the post-transplant treatment costs.

In some embodiments, the closed portion may comprise a visual guide to assist removing the insulating medical device from the graft. The visual guide may be substantially in the middle of the closed portion. The closed portion may include perforations to assist removing the insulating medical device from the graft. The closed portion may include divots to assist removing the insulating medical device from the graft. The closed portion may include a cut line to assist removing the insulating medical device from the graft.

The graft may be a transplantable organ selected from the group comprising kidneys, a heart, a liver, lungs, intestine and pancreas. Other organs not listed here may also be selected and embodiments of the invention can be adapted to any suitable organ type.

In some embodiments the insulating medical device further comprises a Cold Saline (CS) insert along with the silicone material of the curved receptacle. Further, the CS insert is adapted to be cooled while the graft within the cavity is in the cold storage and the CS insert is adapted to keep the graft cool once the medical device is removed from the cold storage. In some embodiments the insulating medical device comprises a cooling pocket such as Cold Saline (CS) pocket that is built into the device during manufacture thereof. This prevents the need to add CS or another cooling insert as this is already contained within the device. In some embodiments the insulating medical device comprises an endothermic reaction fluid pocket that is built into the device during manufacture thereof.

In some embodiments the insulating medical device may further comprise a Polyurethane (PU) insert along with the silicone material of the curved receptacle. Further, the PU insert may be adapted to be cooled while the graft within the cavity is in the cold storage and the PU insert may be adapted to keep the graft cool once the medical device is removed from the cold storage. In some embodiments the insulating medical device may comprise a Polyurethane (PU) pocket that is built into the device during manufacture thereof. This prevents the need to add CS or another cooling insert as this is already contained within the device.

In some embodiments the insulating medical device further comprises straps and respective strap locks connected with the curved receptacle proximal to the open portion, configured to secure the graft within the cavity and keep the graft in contact with the curved receptacle. In some embodiments the insulating medical device may comprise adhesive strips to secure the graft within the cavity and keep the grant in contact with the curved receptacle.

In some embodiments the insulating medical device may further comprise a plurality of cooling pipes in the cavity, the plurality of cooling pipes being connected with a pump and the cold liquid storage at the other end. Further, the plurality of cooling pipes, using the pump, may be configured to continuously provide cold liquid to the cavity to cool the graft and extract the warm liquid obtained after exchanging heat with the graft, to maintain the predetermined temperature range with the cavity.

In some embodiments the insulating medical device may further comprise a Thermoelectric Cooler (TEC) chip disposed in the cavity, the TEC chip being connected with a voltage source. Further, the TEC chip may be configured to remove the heat from the graft using the Peltier effect by creating a heat flux after an application of a voltage from the voltage source, thereby maintaining the graft in the cavity within the predetermined temperature range.

According to another aspect of the invention there is provided an insulating medical device for a graft for transplant, the insulating medical device comprising: a base; sidewalls extending from the base to define a cavity adapted for receiving the graft; the sidewalls having an open portion through which the graft is inserted into the cavity, wherein the sidewalls comprise a biocompatible insulating material and are adapted to snugly hold the graft when placed in the cavity thereby to insulate the graft.

In some embodiments the insulating medical device may comprise a cooling pocket containing Cold Saline and/or Polyurethane and/or endothermic reaction fluid adapted to cool the sidewalls and/or the graft. In some embodiments the cooling pocket may be contained in the sidewalls and/or the base.

In some embodiments the insulating medical device may comprise retaining strips that extend across the open portion for containing the graft in the cavity.

In some embodiments the base may comprise a means adapted to selectively separate the base from the sidewalls thereby to remove the insulating medical device from the graft. In some embodiments the means adapted to selectively separate the base from the sidewalls may comprise one or more of the following: perforations; divots; a cut line adapted for cutting.

In some embodiments the insulating medical device may comprise an active cooling means comprising one or more of the following: cooling pipes connected to a pump; a thermoelectric cooler (TEC) chip connected to a voltage source.

Other aspects are also disclosed.

### BRIEF DESCRIPTION OF DRAWINGS

At least one example of the invention will be described with reference to the accompanying drawings, in which:

FIG. 1A illustrates an insulating medical device for protecting a graft for transplant, in accordance with an embodiment of the present invention;

FIG. 1B illustrates the insulating medical device for protecting a graft for transplant, in accordance with another embodiment of the present invention;

FIG. 2 illustrates an implementation of the insulating medical device of FIG. 1A, in accordance with an embodiment of the present invention; and

FIGS. 3-5 illustrate experimental data of a comparison between the prior art and several embodiments of the present invention, after implementation;

FIG. 6 is a schematic that illustrates a number of example organs and matching embodiments of the present invention;

FIG. 7 illustrates the insulating medical device for protecting a graft for transplant, in accordance with another embodiment of the present invention;

FIG. 8 illustrates another view of the insulating medical device for protecting a graft for transplant, in accordance with the embodiment shown in FIG. 7;

FIG. 9 illustrates experimental data of a comparison between the prior art and the embodiment shown in FIG. 7.

FIG. 10 shows the insulating medical device for protecting a graft for transplant, in accordance with yet another embodiment of the present invention;

FIG. 11 shows the insulating medical device for protecting a graft for transplant, in accordance with another embodiment of the present invention.

It should be noted that the same numeral represents the same or similar elements throughout the drawings.

### DESCRIPTION OF EMBODIMENTS

The present invention provides an insulating medical device that thermally insulates a graft (for example, a kidney implant) prior to and during a transplantation procedure to reduce warm ischaemic injury, reduce time-pressure on the surgeons and medical staff and therefore increase graft survival. The insulating medical device achieves the above-mentioned objective by maintaining the kidney at a safe and cool temperature in the body during transplantation to avoid the warm ischaemic injury occurring during the procedure, prior to revascularization. Potential benefits therefore include increase short- and long-term function, increase patient quality of life, reduce the pressure for rapid surgery (opening the door to robotic transplantation surgery and improved surgical training) and reduce the post-transplant treatment costs.

In this regard the invention below has been discussed with the help of figures for clarity. However, a skilled addressee would appreciate that the invention is not limited to particular types of implementations that have been discussed below and may be equally applicable to many different implementations without departing from the scope of the present invention.

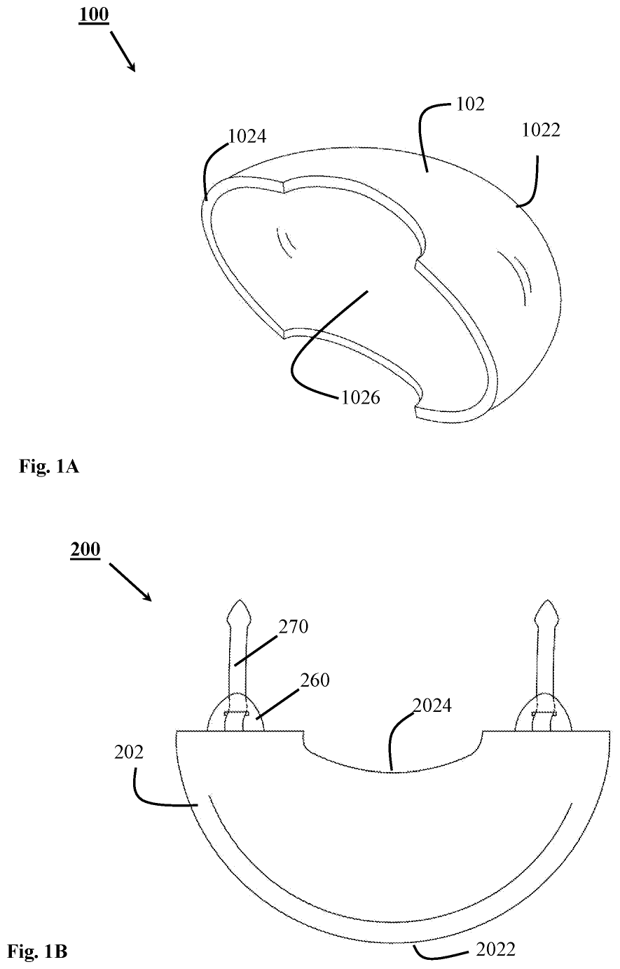

FIG. 1A illustrates an insulating medical device 100 for protecting a graft for transplant, in accordance with an embodiment of the present invention. The graft (not shown) may be a transplantable organ selected from the group comprising, but not limited to: kidneys, a heart, a liver, lungs, intestine and pancreas. As shown in FIG. 1A, the insulating medical device 100 comprises a curved receptacle 102. The curved receptacle provides a thermally insulating cover for the graft, in use. The curved receptacle 102 has an open portion 1024 and a closed portion 1022, that forms a cavity 1026 therebetween. In use the open portion is directed towards the vascular supply to allow anastomosis. The curved receptacle 102 is adapted to receive the graft in the cavity 1026 from the open portion 1024 and secure the graft therein. The shape of the curved receptacle 102 is selected based on a shape of the graft as the curved receptacle 102 must conform to the shape of the graft. For example: in FIG. 1A, the graft is envisaged to be a kidney implant, the curved receptacle 102 is in the shape of a bean similar to the shape of the kidney. In another example, where the graft is a liver or heart implant, then the shape of curved receptacle 102 may resemble a shape of the liver or a heart. In yet another example, the receptable may not have a curved shape but may be made of a material that sufficiently conforms to the shape of the graft when the graft is received therein to provide a snug fit of the graft within the receptacle. For example, the graft may be one or both lungs, heart, kidney, stomach, liver, pancreas or part of the intestines.

The size of the curved receptacle 102 may vary as per the medical application. For example, a length of the insulating medical device 100 shown in FIG. 1A may be in the range of, but not limited to, 170-180 mm. The width may vary from, but is not limited to, 85-95 mm. Similarly, a height may be, but not limited to, between 85 to 95 mm and a thickness may be in between, but not limited to, 2-7 mm respectively. Also, the curved receptacle 102 of FIG. 1A, may be manufactured as an integral unit without any joints. Further, the curved receptacle 102 may be made of any flexible, non-toxic and insulating material such as, but not limited to, silicone. In other embodiments, the receptacle may be made of polyurethane or aliphatic polyester or other suitable material.

This provides an external insulating layer to the graft received in the curved receptacle 102 as well as a firm grip to the medical practitioner holding the insulating medical device 100. Preferably the receptacle 102 is available in a number of different sizes such as small, medium, large, extra-large and are available for selection by medical staff so as to ensure the graft fits snugly within the receptacle to ensure good thermal contact between the graft and the insulating device.

In another embodiment 200 shown in FIG. 1B, the insulating medical device 100 comprises an additional material (not shown) along with the silicone that may be inserted into inner walls of the curved receptacle 102. This helps enable static cooling within the curved receptacle 102. The additional material may be provided in the form of inserts in the curved receptacle 102. In one embodiment, the additional material may be a Cold Saline (CS) insert and in another embodiment, the additional material may be a Polyurethane (PU) insert or endothermic reaction fluid insert. The insulative material may one of the following group but not limited to: solid, a film, foamed, porous, fibrous, crimped, liquid, gaseous, porous, multiphasic, containing a phase that transforms endothermically. In other embodiments, a combination of insulative materials may be used. These materials are selected due to their abilities to retain cooling. Inner walls of the receptacle may include pockets or slots into which the inserts are positioned. In other embodiments the CS, PU or endothermic reaction fluid are built into the curved receptacle 102 at the time of manufacture to prevent the need to provide inserts at the time of the procedure. The inserts can be built into walls of the receptacle 102, for example, by overmolding silicone over inserts. The curved receptacle 102 of the embodiment 200 is fabricated using flat moulds and then joined together with silicone. In other embodiments, the curved receptacle may be made in a single mould and therefore, be of unitary construction. It is envisaged that the curved receptacle can be made in a number of other ways.

In the embodiment shown in FIG. 1B, the insulating medical device 200 further comprises straps 270 and respective strap locks 260 connected with the curved receptacle 202 proximal to the open portion 2024. The straps 270 and strap locks 260 are configured to secure the graft within the cavity 2026 and keep the graft in contact with the inner walls of curved receptacle 202 and the PU or CS inserts, inserted therein. In other embodiments the receptacle is secured using adhesive strips across the open portion. In other embodiments, the graft may be kept secure within the receptacle using other types of fasteners.

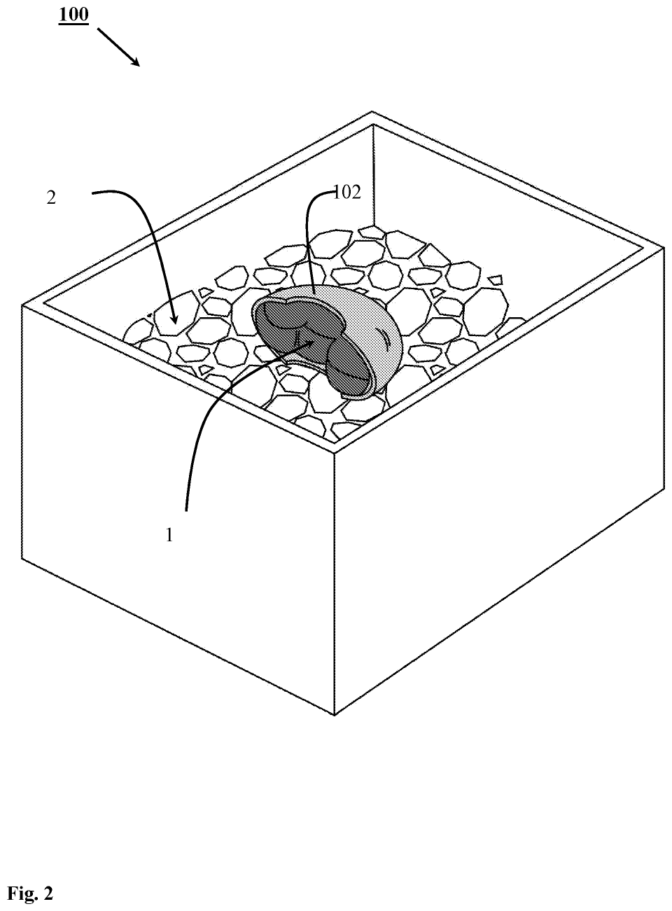

FIG. 2 illustrates an implementation of the insulating medical device 100 of FIG. 1A, in accordance with an embodiment of the present invention. As shown in FIG. 2, the graft 1 is a kidney implant secured in the insulating medical device 100. Prior to the use, the insulating medical device 100 and the graft 1 have been stored in a cold storage 2. The cold storage 2 may be, but not limited to, any ice box, refrigerator or freezer. In the storage the temperature of the graft 1 i.e. the kidney implant in this case, is very low (say, 0-8° C.). So, in prior arts, the graft 1 was taken out from the cold storage 2 without any insulation. The Literature highlights that the kidney implant temperature during transplantation, without insulation, reaches 25-30° C. by the time reperfusion occurs. So, the present invention offers the advantage to the medical practitioner that he/she may take out graft 1 from the cold storage 2, secured within insulating medical device 100. This would prevent a substantial amount of heat transfer from the ambient to the graft 1, that would have otherwise taken place without the insulating medical device 100.

Additionally, the curved receptacle 102 is configured to keep the graft 1 (kidney implant) received therein, within a predetermined temperature range with the help of insulating properties of silicone. The predetermined range may be, but not limited to, 4-25° C., though the present invention keeps the graft 1 within 4-15° C. for as long as possible. This helps to prevent damage to the graft 1 when the graft 1 is removed from a cold storage 2 for transplantation due to rapid rise in temperature.

In the embodiments (implementation not shown in figures) including the CS and PU inserts in the curved receptacle 102, the CS insert is adapted to be cooled while the graft within the cavity 1026, is in the cold storage 2. Then, when the medical device 100 is removed from the cold storage for transplanting the graft, the CS insert is adapted to keep the graft cool till the graft is taken out from the insulating medical device 100. Similarly, the PU insert is also adapted to be cooled while the graft within the cavity 1026, is in the cold storage. And once the medical device 100 is removed from the cold storage for transplanting the graft the PU insert is adapted to keep the graft cool once the medical device 100 is removed from the cold storage. The CS, PU and/or endothermic reaction fluid inserts or built in pockets provide static cooling over a period of time to the graft due to the cooling storage capacity.

In yet another embodiment (not shown), the insulating medical device 100 is connected with an integrated fluid cooling system. The insulating medical device 100 comprises a plurality of cooling pipes in the cavity 1026. The plurality of cooling pipes are connected with a pump and the cold liquid storage at the other end. The plurality of cooling pipes are configured to continuously provide cold liquid to the cavity 1026 to cool the graft using the pump. The cold liquid then comes in contact with the graft and to exchange the heat of the graft. The cold liquid cools the graft while the cold liquid absorbs heat from the graft and is heated in the process. After that the warm liquid obtained after exchanging heat with the graft, is extracted from the cavity 1026. This process goes on continuously to maintain the required temperature range.

In another embodiment (not shown), the insulating medical device is connected with an external fluid cooling system where the fluid is air. A fan or other type of air pump can be used to direct sterilized air towards the cover body with the graft received therein.

In yet another embodiment of the present invention (not shown), the insulating medical device comprises a thermoelectric cooler. In an example, a Thermoelectric Cooler (TEC) chip is disposed in the cavity as a cooling mechanism. The TEC chip may be connected with a voltage source to enable a flow of the current from one side to another. The TEC chip is configured to remove the heat from the graft using the Peltier effect by creating a heat flux after an application of a voltage from the voltage source and flow of current. This helps to maintain the graft in the cavity within the predetermined temperature range.

Another type of heat sink module or bank of modules can be inserted within a pocket with the cavity or attached to an inner surface of the cavity. The heat sink may be made of multi-layered foils of iron, steel copper or gold or multi-layered foils of these metals.

The TEC chip or other heat sink may be connected to a heat exchange or radiator that loses heat energy to the ambient air. Hermetically sealed battery powered units can be used to power the thermoelectric cooler. The cooler can be applied to selective portions of the graft or to the whole graft, in use.

In use, an insulating medical device 100 is cooled in preparation for use as shown in FIG. 2. The graft in this case, kidney 1 is inserted into the cavity 1026 in preparation for the procedure. The kidney is kept cool by the insulating device 100 and any cooling pockets that may be included therein. The device 100 including kidney 1 are then taken for the graft procedure during which the kidney is grafted to the patient while it is still in the device 100. In use, the open portion is directed towards the patient's vascular supply to allow anastomosis. At the completion of the procedure the device 100 is cut along the close portion 1022 so as to remove the insulating device 100. Perforations and/or divots and/or cut lines and/or coloured lines may assist the medical staff to easily cut or separate the closed portion 1022. In some embodiments the closed portion includes a pull tab that actuates perforations to thereby remove the insulating device without the need for scissors or the like that may accidentally damage the patient of the grafted organ.

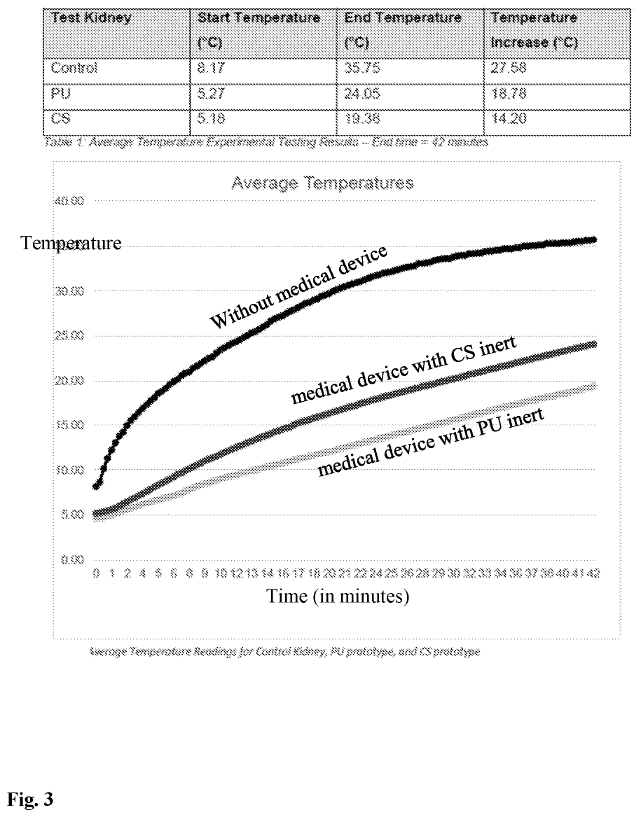

FIGS. 3-5 illustrate experimental data of a comparison between the prior art and several embodiments of the present invention, after implementation. Shown in FIG. 3 is a comparison of the experiment conducted involving a kidney implant without insulation (hereinafter referred to as the “control” kidney), “test” kidney implant in medical device 100 having Cold Saline (CS) insert (referred to as “CS prototype”) and “test” kidney implant in medical device 100 having Polyurethane (PU) insert (referred to as “PU prototype”).

The experimental method is performed on the kidney implant using the following procedure:

1. A water bath is heated to a constant temperature of 37° C. to mimic the body temperature of the patient when the kidney transplant procedure takes place.
2. A “test” kidney is placed inside each of the CS prototype and the PU prototype which is to be tested.
3. A “control” kidney is placed inside the water bath directly to mimic a non-insulated kidney which is the current state of kidneys in kidney transplants today.
4. Three temperature sensing probes are inserted on the anterior side of the kidney, the posterior side of the kidney, and inside the kidney via an incision made to determine its internal temperature changes. This step is performed for both the “test” kidney and the “control” kidney equating to a total of six temperature sensing probes.
5. The temperature sensing probes are connected to an Arduino UNO which is running code that takes measurements after a given time interval (20 seconds by default) and outputs these measurements on a computer screen.
6. The temperature readings are taken for ˜45 minutes (the average time taken to perform a kidney transplant) and plotted on a temperature vs. time graph to observe the cooling efficiency of the embodiment under testing.

As observed in FIG. 3, the control kidney reaches ˜36 degrees after 42 minutes. The CS kidney average temperate is ˜24 degrees after 42 minutes, whereas the PU kidney average temperature is ˜19 degrees. A tabulated summary of these temperatures can be viewed in the accompanying Table.

From FIG. 3, it is evident that both insulation methods (CS and PU) provide better thermal insulation than the control kidney. The insulative (polyurethane) insulative method appears to perform better than the saline insulation, with a difference of ˜5 degrees at 42 minutes.

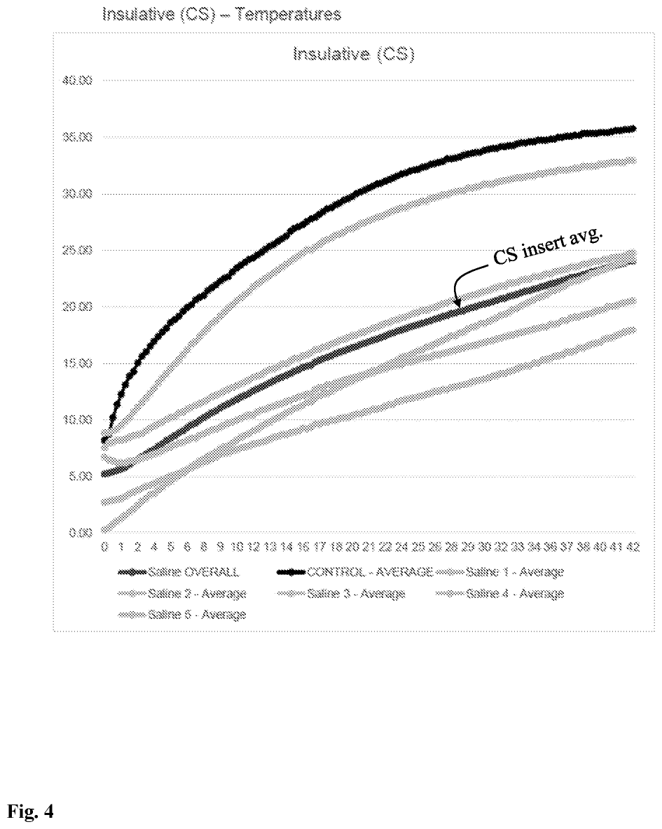

Similarly, a number of iterations of the experiment were conducted separately with each of the CS and the PU prototypes. FIG. 4 illustrates the results of the experiment with CS prototypes. As shown in FIG. 4, the cold saline prototypes can be observed alongside the control kidney temperature (in black). As can be observed, the CS prototypes can be observed to have performed well, with temperatures close to the overall average CS line marked distinctively in the FIG. 4.

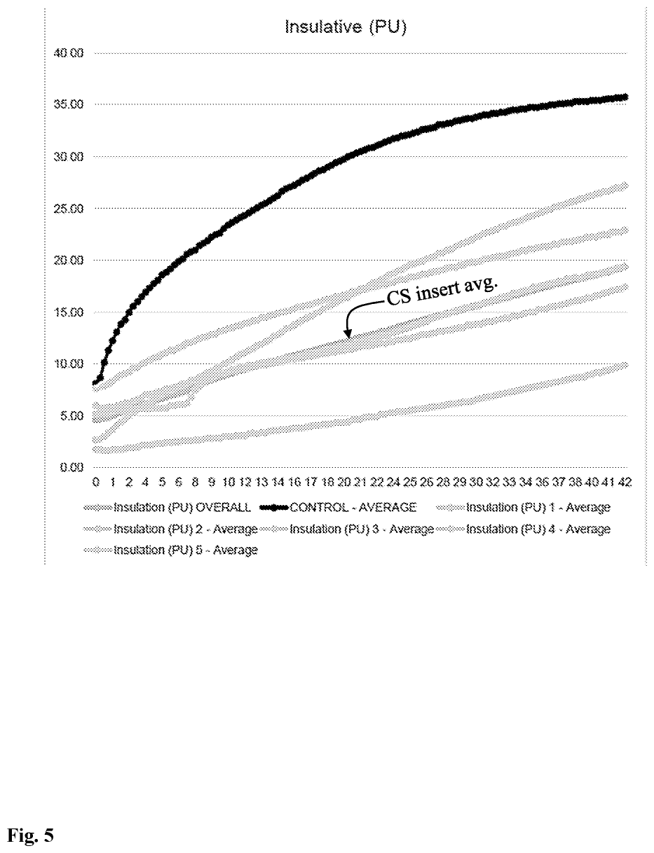

FIG. 5 illustrates the results of the experiment with PU prototypes. From FIG. 5, the polyurethane prototypes can be observed alongside the control kidney temperature (in black). It is visible that the PU prototypes have all performed well and all can be observed close to the overall average PU line marked distinctively in the FIG. 5. When visually compared with FIG. 3, the gradients of the PU prototypes can be observed to be flatter, concluding that the PU prototypes have a slower rate of reheating i.e. a better thermal insulation rate.

From the experimental data, it may be concluded that:
Both embodiments of FIG. 1B (insulation methods involving CS and PU inserts) provide better thermal insulation than the non-insulated method.
Out of PU and CS based insulating medical device 100, the PU prototype is the more effective cooling means after 42 minutes
The CS prototype does reach a lower temperature than the control kidney, however, is not as effective as the PU prototype, and requires freezing before use to ensure the saline inside the silicone is cold which reduces its easy-of-use and accessibility factor.
Furthermore, it is expected that embodiment of FIG. 1A that is fabricated as an insulating medical device 100 made solely out of silicone without any cold saline or PU inserts will be more effective at cooling than other embodiments.

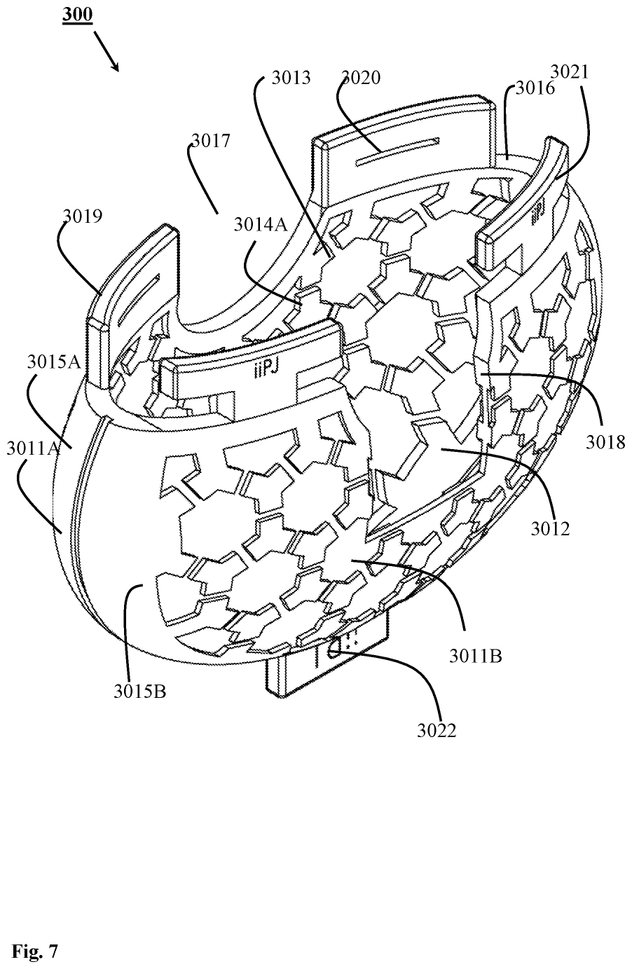

FIG. 7 illustrates another embodiment of the insulating medical device 300. The thermally insulating medical device includes a thermally insulating cover 301. The thermally insulating cover includes a cover body 3011. The cover body defines a cavity 3012 within which a graft is received in use. The cover body 3011 has an opening 3013 through which the graft can be received into the cavity 3012. The cover body 3011 is made of a biocompatible thermally insulating material. In use, the cover body 3011 is configured to keep the graft received therein sufficiently cool to substantially prevent warm ischaemic injury to the graft due to an increase in a temperature of the graft when it is removed from cool storage.

In particular, the cover body 3011 has an inner surface 3014 that defines the shape of the cavity 3012. The cavity 3012 has a shape that is similar to a shape of the graft so as to closely cover the graft, in use. In this embodiment, the cover body 3011 is similar to a shape of the graft. The cover body 3011 also has a substantially uniform thickness to evenly provide insulation across the graft.

The cover body 3011 is curved. The cover body 3011 comprises a first portion 3011A and a second portion 3011B opposed to each other, each portion being similar in curvature. Each portion has an inner surface 3014A, 3014B and an outer surface 3015A, 3015B. The inner surfaces 3014A, 3014B of the portions are in contact with outer surfaces of the graft in use.

The cover body 3011 may be made of a flexible material that conforms to an outer shape of the graft, in use. This reduces the likelihood of ambient air circulating between inner surfaces of the cover body 3011 and the outer surfaces of the graft. This reduces heat transfer into the graft via thermal diffusion.

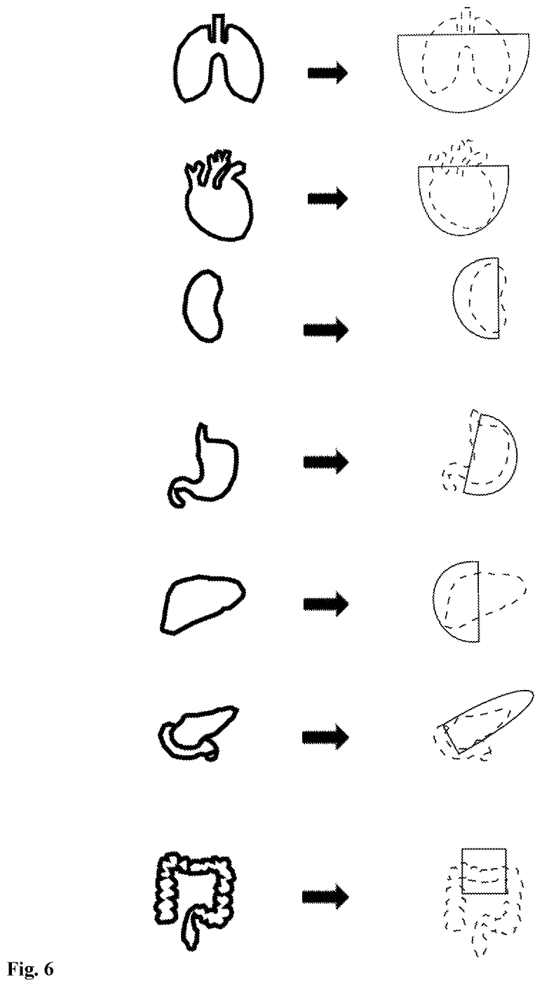

As mentioned above, FIG. 6 illustrates a number of different graft organs and matching embodiments of the thermally insulating medical device. For example, the graft may be lungs, heart, kidney, stomach, liver, pancreas, part of the intestines or other organ or part of an organ. In this embodiment, the graft is a kidney.

The cavity 3012 within the cover body 3011 is shaped and sized such that a substantial proportion or substantially all of an external surface area of the graft can be covered by the cover body 3011, in use.

The cover body has a substantially uniform thickness. The thickness of the cover body is 2 cm. In other embodiments, the thickness of the cover body can be less than 2 cm or more than 2 cm. In other embodiments, the thickness of the cover body can be within the range of 1.5 cm to 2 cm.

As mentioned above, the cover body 3011 has an opening 3013 through which the graft is received into the cavity 3012. The shape of the opening is defined by an outer edge 3016 of the cover body. The opening 3013 is shaped and sized to receive the organ into the cavity 3012 without unnecessarily applying pressure to parts of the graft which may damaging the graft.

The cover body 3011 has a curved recess 3017 extending into the cover body from the outer edge 3016. The curved recess 3017 is configured to allow the blood vessels and the ureter which extend outwardly from near the centre of the kidney to remain uncovered. Conveniently, the surgeon can perform the transplantation surgery to connect the graft kidney to the patient while the graft is within the cavity 3012 of the cover body and, therefore, thermally insulated.

In this embodiment, there is a curved recess 3017 extending into one curved portion of the cover body 3011A from the outer edge and a second, rectangular recess 3018 extending into the other curved portion of the cover body 3011B. The second recess 3018 is directly opposite the curved recess 3017.

In another embodiment, the first recess and the opposed second recess may be identical and curved. In this embodiment, as the recess is curved in an arc about a central axis, the cover body is also curved in an arc about the same central axis and has a U-shaped cross section. The two arms of the U extend substantially parallel to a plane perpendicular to the central axis.

The second, rectangular recess 3018 provides a window for the surgeon to view the organ while it is within the cover body, in use. This will allow the surgeon to assess and diagnose potential complications with the graft kidney during surgery while it is within the thermally insulating cover.

The embodiments illustrated in FIGS. 7, 8, 10 and 11, the internal and external surfaces of the cover body 3014A, 3014B, 3015A, 3015B are textured. In these embodiments, a pattern 3023 is debossed on each of the inner surfaces of the two portions of the cover and the same pattern is embossed on each of the external surfaces of the two portions. In this way the external surfaces of the two portions are negatives of the internal portions of the two surfaces.

To create the cover body 3011, the moulds can include negatives of the patterned surfaces. Thus, the formed cover body 3011 will have the pattern on external surfaces of the two portions of the cover body 3011A, 3011B. It is envisaged that in other embodiments different types of patterns can be created on the surfaces of the cover body in various different ways.

Advantageously, the textured inner surfaces 3014A, 3014B provide a gripping surface to outer surface of the graft which prevents egress of the graft from the cavity, in use.

The textured surfaces 3014A, 3014B, 3015A, 3015B provide an interconnected network of cooling channels which allow cooling fluid such as cold saline to be distributed along an external surface area of the cover body and thereby, cool the cover body 3011 and graft received therein. During surgery, cold saline poured onto the external surfaces 3015A, 3015B of the graft will travel along channels within the pattern to cool the graft. The pattern may also include relatively flat areas for cooling liquid to pool.

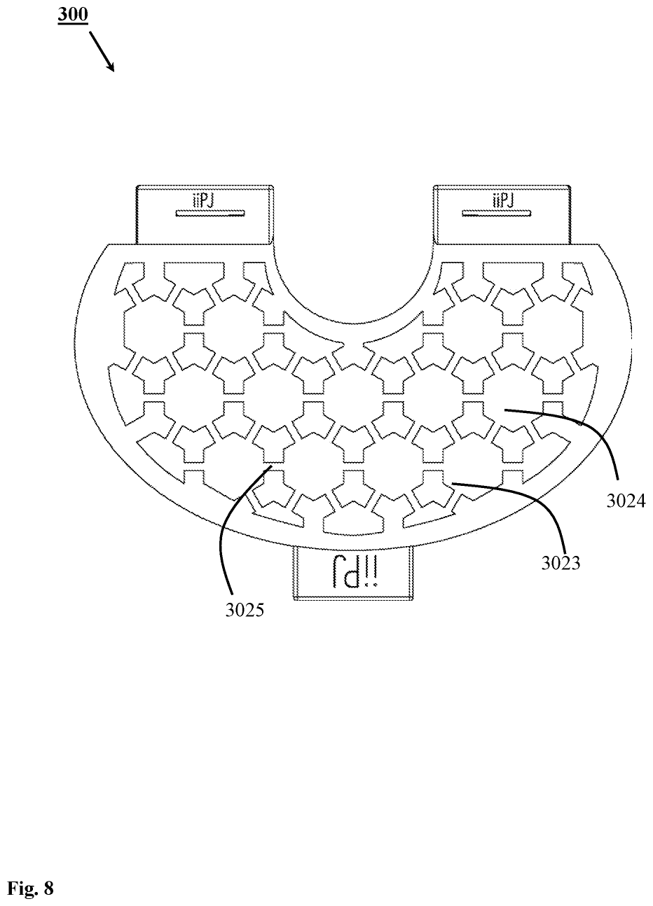

The embodiment shown in FIGS. 7 and 8 have a network of interconnected hexagons 3024 evenly distributed over the surfaces of the cover body. There are channels 3025 between adjacent hexagons. Each of the hexagons 3024 and channels 3025 are recessed into the surface which allows the cooling liquid to pool within the hexagons 3024 to cool the graft through the insulating layer.

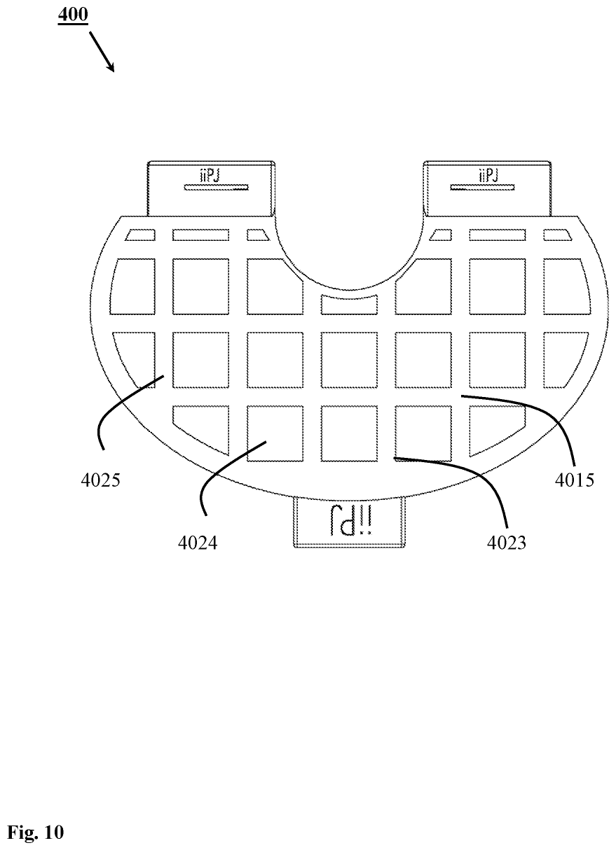

The embodiment in FIG. 10 has a pattern 4023 comprises a plurality of squares 4024. The squares of the plurality of squares 4024 are separated from each other via an interconnecting network of channels 4025 on the outer surface 4015.

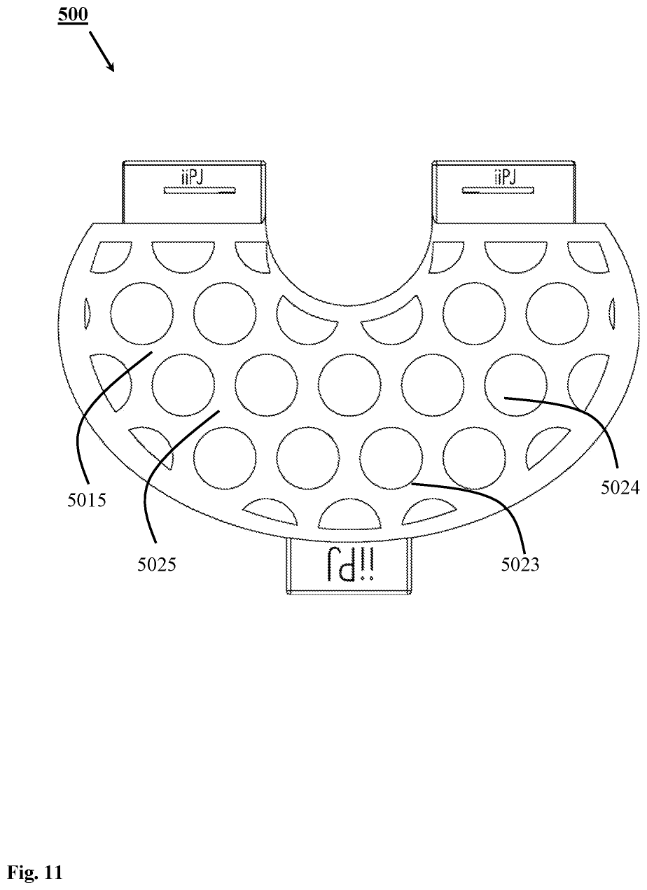

The embodiment of FIG. 11 has a pattern 5023 comprising a plurality of circles 5024 evenly distributed across the cover body surrounded by interconnected spaces 5025 recessed into the cover to allow pooling of cooling fluid on the outer surface 5015.

Advantageously, the textured surfaces can create air pockets between the graft and the cover body, for example, where a part of the surface is higher than another adjacent part of the surface, to enhance cooling of the graft by providing extra insulation.

It is envisaged that the textured surfaces can be created in a number of ways for example, surfaces of the moulds used to make the cover body may be patterned.

The thermally insulating cover shown in FIGS. 7 and 8 also includes two fasteners 3019 for securing the graft within the cavity. When fastened, each fastener is configured to provide a barrier across the opening to prevent egress of the graft from the cavity when a user is manipulating or moving the cover body. Each fastener 3019 is located adjacent either side of the recess so as not to interfere with blood vessels extending out of the recess, in use.

The fastener 3019 shown in FIG. 7 has a first portion 3020 and a corresponding, second portion 3021. The first portion of the fastener 3020 is attached adjacent to a part of the outer edge 3016 at the first portion of the cover body. The second portion of the fastener 3021 is attached at a corresponding position across the opening, adjacent a part of the outer edge 3016 of the second portion of the cover body.

In this embodiment, the first portion of the fastener 3020 comprises a horizontal slot within a rectangular projection and the second portion of the fastener 3021 comprises a T-shaped member which engages with the slot in the first portion 3020. The horizontal component of the T-shaped member is longer than that of the slot so that the T-shaped member can be retained within the slot. Advantageously, the cover body 3011 and such fasteners 3021 can be manufactured at the same time via the same manufacturing process.

It is envisaged that a number of other types of fasteners can be used such as biocompatible velcro, biocompatible adhesives, pins, buttons and clips.

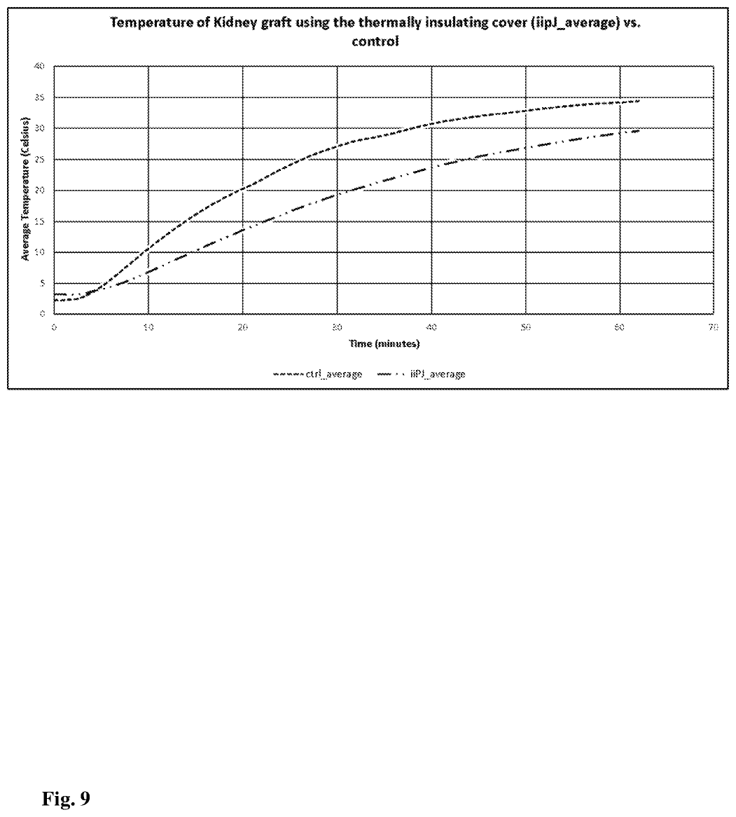

FIG. 9 illustrates the thermal performance of the thermally insulating cover shown in FIGS. 7 and 8, relative to an uncovered graft when both are removed from cold storage and placed in a water bath at a temperature of 35 degrees. The average temperature of the graft is notably lower than that of the uncovered graft over 60 minutes. This is advantageous as typically most kidney transplants take between 45 minutes to 60 minutes.

In use during surgery, after being removed from cool storage, the insulating cover with the graft received therein can be placed in contact with a cooling medium e.g. a bed of ice. The insulating cover will assist the graft in retaining cooling. Advantageously, the insulating cover slows the rate of heat transfer into the graft, thus assisting in preventing warm ischaemic injury over time. Furthermore, there will be a reduced need to actively cool the graft during surgery by, for example, monitoring a temperature and/or providing cooling to the graft via an external source. Thus, the insulating cover provides a passive cooling effect to the graft in ambient temperature relative to an uncovered graft.

In other embodiments, the cover body 3011 can include slots or other formations for securing the graft within the cavity by narrowing or closing the opening with surgical clamps or other surgical tools.

The thermally insulating cover 300 may include a gripping protrusion 3022 located between the first and second portions of the cover body 3011A, 3011B. The gripping protrusion 3022 may be rectangular in shape and sufficiently wide to allow a user to comfortably grip and manipulate the cover body 3011 including the graft secured therein.

In this embodiment, the gripping protrusion 3022 is located on a part of the cover body 3011 that is opposite the opening and in particular, opposite the location of the recess.

In other embodiments, there may be multiple gripping protrusions located on the cover body for the user to grip and manipulate the cover body 3011 including the graft, during surgery.

The cover body 3011 may also have markings which provide a guide for the user during surgery. The cover body may also have a perforated line located along a join between the first and second portions of the cover body 3011A, 3011B. In use, the perforated line acts as a cutting guide to allow controlled disassembly of the cover body 3011 as described above.

In other embodiments markings can be visual indicators for anatomy, orientation, identification and/or analysis during transplantation. The markings can be coloured, textured, have symbols and/or labels. The indicators may highlight key anatomical areas of interest, important vessels and structures and provide guidance for surgical cuts and sutures.

In another embodiment, the thermally insulating medical device can also include an adjustable cover which can be used to temporarily uncover exposed organ tissue. As surgery is conducted, adjustable covers over parts of the main body can be moved, added or removed in order to cover or to expose any tissue that may or may not be located within the cover body.

In other embodiments, the thermally insulating medical device may comprise an outer layer of a thermally conductive material on outer surfaces of the cover to reflect thermal radiation. The thermally conductive material can be a metal film. The thermally conductive material may be sputter coated via other types of physical vapour deposition. The thermally conduct material may also be applied to the cover via chemical vapour deposition. A thickness of the film can range between 5 to 800 nm. The film thickness can be 10 nm thick. Alternatively, the metal layer may be in the form of a fibrous sheet integrated with or removably attachable to outer surfaces of the cover body.

The thermally insulating medical device can be used to selectively cool tissue during surgery to prevent thermal injury to tissues surrounding a surgical zone. For example, the thermally insulating medical device can be used to provide cooling to tissues or implanted medical devices such as pacemakers or other medical devices which stimulate tissue, adjacent tissue that is being treated during surgery. The surgery may be a type of electrosurgery in which heat is applied to tissue such as that uses electromagnetic radiation), electrocautery, ultrasound cutting or ablations using laser ablation tools.

The thermally insulating medical device can also be used to cool surgical tools such as drills, blades and reamers before they are used to limit injury to tissues surrounding the tissue that is being operated on. The chilled metal tools will draw heat from cutting surfaces to prevent the likelihood of tissue necrosis in areas surrounding the tissue. Cooling the cutting tool and tissue zone may also help limit blood loss during surgery and subsequent implant loosening and risk of infection.

For example, the biocompatible thermally insulating material may be silicone, or polyurethane or an aliphatic polyester. In this embodiment, the biocompatible thermally insulating material is silicone. Advantageously, silicone can be sterilized using current methods in the art without being damaged during the sterilization process.

The present invention offers a number of advantages. Firstly, the present invention can be provided as an off-the-shelf, single use, sterile medical device that is biocompatible as well as intuitive and easy-to-use. Within hospitals with transplant units the present invention could be provided as a one-off disposable to be utilised in all organ transplantation procedures to prevent warm ischemic injury, remove the pressure for fast surgery and to provide additional time for surgical training Additionally, the present invention presents significant cost saving when accounting for the health economic effects associated with graft failure (increased dialysis, longer patient stays, graft rejection etc.). The present invention does not require any prior treatment of the graft (organ implants) other than normal static cold storage which is a gold standard in organ transplantation. Furthermore, the present invention can be manufactured according to the graft shape so that the receptacle or insulating cover conforms around the whole organ implant while giving access to important vascular or other anatomical regions of interest to the user. Additionally, in the embodiments shown in FIGS. 1A, 7, 8, 10 and 11, the present invention does not require any external apparatus for its functionality.

Various modifications to these embodiments are apparent from the description to those skilled in the art. The principles associated with the various embodiments described herein may be applied to other embodiments. Therefore, the description is not intended to be limited to the embodiments but is to be providing broadest scope consistent with the principles and the novel and inventive features disclosed and/or suggested herein. Accordingly, the invention is anticipated to hold on to all other such alternatives, modifications, and variations that fall within the scope of the present invention.

1. A medical device for thermally insulating a graft to be transplanted, comprising:
a cover body including an inner surface defining a cavity within which the graft is received, in use, the cover body having an outer edge defining an opening through which the graft is received into the cavity, in use;
wherein the cover body is made of a biocompatible thermally insulating material;
wherein, in use, the medical device is configured to keep the graft received therein sufficiently cool in the body of a subject during surgery to substantially prevent warm ischaemic injury to the graft.

2. The medical device of claim 1, wherein an external surface of the cover body is biocompatible.

3. The medical device of claim 1, wherein a shape of the inner surface of the cover body is similar to a shape of the graft.

4. The medical device of claim 1, wherein the graft is a transplantable organ selected from the group comprising kidneys, a heart, a liver, lungs, intestine and pancreas.

5. (canceled)

6. The medical device of claim 1, wherein the cover body extends over substantially all of an external surface area of the graft, in use, and/or the cover body is adapted to closely cover the graft, in use.

7. (canceled)

8. The medical device of claim 1, wherein the cover body is adapted to conform to an outer shape of the graft, in use.

9. The medical device of claim 1, wherein the cover body is made of a flexible material.

10. (canceled)

11. The medical device of claim 1, wherein the cover body has a substantially uniform thickness.

12-13. (canceled)

14. The medical device of claim 1, wherein the cover body comprises an outer surface, the outer surface being textured.

15. The medical device of claim 1, wherein the cover body comprises an inner surface, the inner surface being textured.

16. The medical device of claim 15, wherein the textured inner surface is configured to grip an outer surface of the graft, in use, to substantially prevent egress of the graft from the cavity.

17. The medical device of claim 14, wherein the textured surface comprises a plurality of channels along which cooling fluid can travel to cool an outer surface of the graft, in use.

18-19. (canceled)

20. The medical device of claim 1, wherein the medical device further includes a gripping portion extending from the cover body to allow the user to comfortably grip and manipulate the cover body when the graft is located within the cover body, in use.

21. The medical device of claim 1, further comprising at least one fastener located on the cover body, adjacent the opening, configured to provide a barrier across the opening of the cover body to prevent egress of the graft from the cavity, in use.

22. The medical device of claim 21, wherein the at least one fastener includes a first portion attached adjacent a first portion of the edge, and a second portion adjacent an opposing second portion of the edge located across the mouth.

23. (canceled)

24. The medical device of claim 1, wherein the medical device is of unitary construction.

25-26. (canceled)

27. The medical device of claim 1, wherein the cover body further includes markings which provide a visual guide to the user, in use, and/or further comprising a cooling pocket for receiving a cooling insert.

28-31. (canceled)

32. A medical device for thermally insulating a graft kidney to be transplanted comprising:
a curved cover body having a substantially U-shaped cross-section, the U-shaped cross-section defining a cavity configured to receive and closely cover the graft kidney, in use;
wherein the curved cover body comprises a biocompatible thermally insulating material and is configured to keep the graft kidney sufficiently cool in the body of the subject to substantially prevent warm ischaemic injury to the graft kidney, in use.

33-36. (canceled)

37. The medical device of claim 32, wherein the cover body is curved in an arc, thereby defining a recess such that, in use, blood vessels and ureter of the kidney can be positioned within the recess to enable a user to easily access to blood vessels and ureter of the kidney during transplantation.

38. (canceled)

39. A system for cooling a graft before transplant, comprising:
a medical device for thermally insulating a graft to be transplanted according to claim 1;
an active cooling apparatus configured to provide cooling to the cover and the graft received therein.

40-44. (canceled)

45. A system for cooling a graft before transplant, comprising:
a medical device for thermally insulating a graft to be transplanted according to claim 32;
an active cooling apparatus configured to provide cooling to the cover and the graft received therein.
